(19) United States
(12) Patent Application Publication
(10) Pub. No.: US 2012/0065521 A1
(43) Pub. Date: Mar. 15, 2012

(54) NEEDLE BIOBSY IMAGING METHOD

(75) Inventors: Rebecca Richards-Kortum, Houston, TX (US); Timothy J. Muldoon, Houston, TX (US); Konstantin Sokolov, Austin, TX (US); Jordan Dwelle, Austin, TX (US)

(73) Assignee: The Board of Regents of The University of Texas System, Austin, TX (US)

(21) Appl. No.: 13/229,059

(22) Filed: Sep. 9, 2011

Related U.S. Application Data
(62) Division of application No. 11/464,777, filed on Aug. 15, 2006, now abandoned.
(60) Provisional application No. 60/708,301, filed on Aug. 15, 2005.

(51) Int. Cl. A61B 1/07; A61B 1/04; A61B 6/00

(52) U.S. Cl. 600/476; 600/109; 600/181; 600/182

(57) ABSTRACT

Imaging techniques. Radiation is directed from a source onto a sample using an endoscope having cellular or subcellular resolution. The endoscope includes one or more fibers. The fibers have a proximate end and a distal end, and the distal end is lensless. A focal plane of the endoscope is substantially at a tip of the distal end. Radiation from the sample is directed onto a detector to diagnose or monitor the sample.

### CROSS-REFERENCE TO RELATED APPLICATIONS

This application is a divisional of co-pending U.S. patent application Ser. No. 11/464,777 filed Aug. 15, 2006, which claims priority to U.S. Provisional Application No. 60/708,301 filed Aug. 15, 2005. The entire text of each of the above applications is specifically incorporated herein by reference without disclaimer.

### STATEMENT AS TO RIGHTS TO INVENTIONS MADE UNDER FEDERALLY-SPONSORED RESEARCH AND DEVELOPMENT

Aspects of this invention were made with government support of the NSF, Project Title “NSF IGERT,” Grant No. 0333080. Aspects of this invention were made with government support of the NIH, Project Title “Fiber Optic In Vivo Confocal Microscopy,” University of Texas Account No. 26-1606-76xx.

### BACKGROUND OF THE INVENTION

1. Field of the Invention

The present invention relates generally to the fields of imaging and diagnostic imaging. In one example embodiment, it concerns an endoscope which can be used as an optical needle biopsy to image a layer of cells that are in contact with, or close proximity to, the distal tip of the endoscope. In one example embodiment, the endoscope may comprise a fiber optic image guide. In another example embodiment, the endoscope may comprise a graded-index lens (GRIN) image guide. In another example embodiment, the endoscope may comprise an image guide that comprises both fiber optics and a graded-index lens. In another example embodiment, the endoscope is Magnetic Resonance Imaging (MRI) compatible and can be used for simultaneous MRI and optical imaging.

2. Description of Related Art

Many techniques exist for the detection of cancer and other tissue abnormalities. These techniques often depend upon noticeable changes in the physical, molecular, or metabolic (as well as other) qualities associated with a group of cells.

The hallmark of cancer is uncontrolled and unchecked cell replication. Due to abnormal amounts of DNA replication, nuclei of dysplastic cells can appear greatly enlarged, often comprising 90% of the cell's diameter. These nuclei often appear irregular and hyperchromic because of this abnormal DNA replication. Additionally, because of the high rate of mitosis, numerous mitotic figures may be present in dysplastic tissues. As dysplastic cells divide more frequently, cells will appear crowded and push into locations where they do not normally reside. For example, in normal epithelial tissue, such as skin, there is a clear hierarchy of organization with a basal cell layer that divides to replenish cells above that are sloughed off. A dysplastic lesion in epithelial tissue can be graded based on what fraction of the epithelium has been replaced with the abnormal cells. Higher grade lesions will involve progressively greater fractions of epithelium. If a lesion encompasses the entire epithelium but does not go beyond the basal layer of cells or past the basement membranes, this condition is termed carcinoma in situ. When abnormal cells can be seen to push beyond the basal cell layer and basal membrane and into the connective tissue beneath, malignant transformation is said to have taken place, and treatment for such a lesion will become significantly more aggressive.

Analysis of the histology of removed tissues by a pathologist is the accepted standard of care for making a definitive diagnosis of cancer. The morphologic clues that can be used to aid the pathologist in making a diagnosis are described above. In recent years, however, additional tools have become available to improve the ability of the physician to make diagnoses that are based on the molecular and metabolic features of some types of cancers. Certain breast carcinomas have been shown to overexpress an extracellular tyrosine kinase receptor known as Her-2/neu. This receptor is involved in an estrogen signaling pathway and has been shown to be important in determining the sensitivity of the cancer to a specific type of treatment. Through a process known as immunohistochemisty (IHC), antibodies directed against this receptor can be introduced into the tissue, highlighting regions that express the abnormal receptor. Such molecular-based strategies enables for more specific diagnoses and highly directed treatments based on the expression of such markers. In addition to antibodies, aptamers (short sequences of RNA that have been shown to bind proteins) have been used recently as targeting agents directed against certain receptors.

In addition to the visible changes that occur in tissues due to dysplasia and cancer, there are numerous molecular and metabolic changes that occur as well. Mitotically active cells require a large amount of resources to be able to maintain such a high rate of replication; as a result, their oxygen and nutrient demands are very high. Blood flow to fast-growing tumors is often increased, and frequently associated with abnormal angiogenesis. This increased blood flow and altered metabolic activity within cells can be detected using various spectroscopic techniques.

Despite the ability of these techniques to elucidate functional properties of tissues, there exists a need to evaluate tissues at higher resolution. Spectroscopic methods are unable to resolve tissues down to the cellular level, and are therefore not able to differentiate between malignant neoplasias and certain other benign conditions, such as inflammation. Short of performing a surgical biopsy, several techniques exist that can examine tissue at high resolutions while being only minimally invasive. Needle biopsy is a common technique that can access virtually all parts of the body. In its simplest form, a needle biopsy involves the insertion of a small hollow needle into a suspicious tissue, guided either by palpation, ultrasound, computerized tomography (CT) or other imaging modality. Suction is applied via negative pressure from a syringe at the opposite end of the needle to remove cells from the tissue undergoing the biopsy. These cells can be fixed immediately and stained to enable a fast diagnosis, and may also be saved for more specific studies to assess the specific nature of any tumor cells found, analogous to IHC as described above. While this procedure does not require general anesthesia and surgical complications are minimal, several passes may be required to attain enough cells for a proper diagnosis.

Needle biopsies are usually performed when the nature of a lump, mass, or other area is in question. The biopsies can also be performed on a known tumor or area to assess the effect of treatment or to obtain tissue for other studies. Biopsies are usually done by a trained medical professional assisted by a cytopathologist. A typical procedure involves the insertion of a fine needle, which removes cells or other material from a tumor or mass. More than one needle may be used. For example, one needle may be serve as a guide, while one or more other needles can be placed along it to achieve more precise positioning. After a needle is placed properly, cells may be withdrawn by aspiration with a syringe and placed into a special container. The removed cells are then examined by the cytopathologist, who will attempt to make a diagnosis or provide information necessary for a diagnosis.

Although needle biopsies have several advantages, several drawbacks exist. As with any conventional biopsy, a patient must often endure multiple waiting periods between suspicion, removal, and diagnosis. Time is required for the removal of tissue, histological slicing and staining, and analysis at a pathology lab before diagnosis can be made. Another problem, associated with biopsies used to effect a treatment, involves the need to let biopsy sites heal between biopsies, which makes ongoing treatment monitoring difficult. Additionally, since cells removed via biopsy are removed from their surroundings, the architecture of the tissue cannot be visualized, making it more difficult to perform a pathologic diagnosis.

Other diagnostic techniques involve distinguishing dysplastic tissues from normal tissues through the use of an imaging modality. However, these techniques are dependant upon a chance in contrast between the two tissue types. Fortunately, there are native contrast variations that can be visualized with minimal additional processing. Increased DNA synthesis in the nuclei of dysplastic cells renders their nuclei large, hyperchromic, and highly reflective. This increased reflectivity of dysplastic nuclei has been exploited in reflectance-based imaging techniques to highlight suspicious areas in tissues.

Cervical cancer screening has taken advantage of this concept for many years through the use of colposcopy. This imaging technique uses a relatively low power microscopy to visualize the epithelium of the cervix. While a dysplastic lesion may not always be readily apparent, a weak solution of acetic acid can be applied, which enhances the contrast between normal and abnormal epithelium. The mechanism of this reversible process is not well understood, but likely involves the clumping of chromatin, which in turn further enhances the reflectivity of nuclei by increasing the refractive index mismatch between the nuclei and cytoplasm. This leads to an increase in backscattered light from the tissue. This effect causes dysplastic tissue, with its greater chromatin content, to reflect more light than its surroundings, improving the chances that a clinician will be able to observe the lesion and take the appropriate steps to secure an accurate diagnosis.

In addition to acetic acid, other cancer-specific contrast agents have undergone clinical study. One such compound is toluidine blue, a metachromatic dye that can easily be applied to epithelial tissues. It has been theorized that this dye binds to negatively-charged chromatin in the nuclei of cells, thereby preferentially staining the nuclei of cells that have become dysplastic. The result is not dissimilar from what is seen with the acetowhitening effect: dysplastic or malignant lesions stand out from the background of normal epithelium, alerting a clinician that further study is possible. A visual inspection of the oral cavity using Toluidine blue takes only a few minutes and utilizes reagents that are readily available and inexpensive. Another non-targeted dye, cresyl violet, has also shown promise as an inexpensive marker that is selective for dysplastic and cancerous tissues. It has the added benefit of being fluorescent, simplifying the design of imaging systems intended to work with this dye.

While these techniques are inexpensive and allow for rapid screening, their specificities are not sufficient enough to entirely replace biopsies. Toluidine blue, for example, has been shown to have a high false-positive rate as well. These methodologies still need to be able to demonstrate a high degree of sensitivity and specificity. With the advent of molecular targeting strategies it is possible to achieve acceptable levels of specificities in a number of diagnostic strategies.

Immunohistochemistry (IHC), as discussed previously, has been shown to target with a high degree of specificity abnormal cells that express certain proteins. These proteins can be isolated and produce antibodies that are specific only to these molecules. With the use of these antibodies, diagnostic tests can be specifically designed to detect certain types of cancers that express these specific molecules. Such molecules may be cell membrane localized receptors, secretory products like matrix metalloproteinases, abnormal cell signaling proteins, or a host of other classes of intracellular and extracellular proteins. For diagnostic tests that rely on imaging modalities, the ability to link these highly specific antibodies to markers that enhance the contrast of dysplastic regions is desirable. These markers can be either highly reflective or absorbing for reflective based imaging strategies or fluorescent for fluorescent imaging modalities. Gold nanoparticles have shown much promise in reflectance imaging, as they are easily linked to antibodies or other targeting molecules and exhibit desirable reflectance properties under the right conditions.

Early detection and removal of cancerous tissues has been shown to almost universally reduce the morbidity and mortality associated with the disease. Unfortunately, while a biopsy is usually a very specific technique to determine the pathologic nature of the tissue, the indicators for taking one may sometimes be misleading. For example, benign leukoplakia on the oral mucosa can easily be confused with the clinical appearance of precancer (dysplasia) or that of squamous cell carcinoma. A physician may defer a biopsy from such a location because he or she believes it to be only a benign lesion, delaying treatment.

As a result of the issues associated with the procedures described above, it is sometimes desirable to perform other visual diagnostic procedures. While magnetic resonance imaging (MRI) and computerized tomography (CT) are two widely accepted noninvasive imaging techniques, they are limited in their resolving power and are generally not able to distinguish cancerous from benign tissue at a cellular level. Additionally, CT has the disadvantage of delivering a moderate dose of ionizing radiation to patients.

Standard microscopy generally does not work well on in vivo tissues because of the inherent turbidity present. Since tissue is highly scattering, light from outside the focal plane of interest will be present in the image plane of any microscope device. With standard histopathology, tissue is sliced to form an extremely thin film, whereby essentially all of the material to be observed can be effectively focused. For imaging in live tissues, a technique known as optical sectioning has been shown to provide detailed structural data without needed to physically section tissues.

Confocal microscopy has been used for a number of years for in vitro applications, but has also been shown to be useful for the imaging of ex vivo biopsies and in vivo tissues. A confocal microscope works by focusing the illumination on a small point within the plane of interest. The returning light, which may be either reflected light or fluorescent light, is then focused through a small pinhole at the conjugate image plane. A photodetector placed just behind this pinhole serves to collect this incident light. Light that returns from outside the focal plane of interest is then rejected by the outside of the pinhole, thereby reducing the out of focus scattered light that may otherwise be collected. To create a full image, the illumination is scanned across the entire desired X-Y plane of the frame. The final image lacks the color of a histopathology slide, and is dependant upon refractive-index mismatching (in the case of reflectance imaging) to elucidate nuclei from cytoplasm or other structures. Despite the advantages of confocal imaging, there are drawbacks inherent in its design that limits the potential applications. For example, since illumination must be directed into the tissues and recollected, the penetration depth of confocal microscopy is limited by how deeply the light can pass into tissues. While longer wavelengths of light tend to scatter less and penetrate more deeply into tissues, even near infrared light (NIR) systems can only image to a depth of about 1,000 microns effectively. Additionally, while miniaturization of confocal systems in recent years has created progressively smaller instrumentation, including a confocal endoscope, the optical and mechanical elements of these systems have generally limited the usefulness of this technique to easily accessible regions of the body.

It is therefore desirable to provide optical diagnostic apparatus and procedures without the inherent issues associated with known devices and methods.

These example shortcomings are not intended to be exhaustive, but rather are among many that tend to impair the effectiveness of previously known techniques concerning biopsies. The techniques appearing in the art have not been altogether satisfactory, and a significant need exists for the techniques described and claimed in this disclosure.

### SUMMARY OF THE INVENTION

Certain shortcomings of the prior art may be reduced or eliminated by the techniques disclosed here. These techniques are applicable to a vast number of applications, including but not limited to any application involving the imaging of tissue, and particularly applications that would conventionally call for a needle biopsy.

In one embodiment, a lensless fiber optic endoscope is used as an optical needle biopsy to image a layer of cells that are in contact with, or substantially in contact with, the distal tip of the endoscope. It may be used to, e.g., detect the presence of fluorophores that have labeled individual cells. In conjunction with targeted fluorophores, it is a powerful tool to immediately, or nearly immediately, detect the presence of cancer and other tissue abnormalities in vivo. One may use this technology to image tissue reflectance or other contrast agents as well. In vivo detection of many types of diseases is possible with the device. Additionally, the device is a valuable tool allowing researchers to monitor one site through time to reduce variability of specimen and to reduce the cost of experimentation.

In one respect, embodiments of the invention involve an imaging endoscope apparatus including one or more optical fibers, optics, and a detector. The fibers have a proximate end and a distal end. An optical system directs source radiation to the fibers and an emission from the sample to the detector. Embodiments may comprise a distal end of the fibers that does not include a focusing lens. A focal plane of the endoscope is substantially at a tip of the distal end, and the endoscope achieves cellular or subcellular resolution.

In another respect, embodiments of the invention involve an imaging endoscope including a source of radiation, a collimating lens, a beam splitter, an objective lens, a fiber bundle, a lens, and a detector. The fiber bundle has a proximate end and a distal end, and the distal end does not include a focusing lens. A focal plane of the endoscope is substantially at a tip of the distal end. The endoscope achieves cellular or subcellular resolution.

In another respect, embodiments of the invention involve a method of imaging. Radiation is directed from a source onto a sample using an endoscope having cellular or subcellular resolution. The endoscope includes one or more fibers. The fibers have a proximate end and a distal end, and the distal end does not include a focusing lens. A focal plane of the endoscope is substantially at a tip of the distal end. Radiation is directed from the sample onto a detector to diagnose or monitor the sample. The sample may include human tissue. The sample may include one or more markers or contrast agents. A non-limiting list of example contrast agents and markers includes toluidine blue, cresyl violet, acetic acid, fluorescein, NBDG (a fluorescent glucose analog), antibody-targeted fluorescent dyes, antibody-targeted nanoparticles, antibody-targeted quantum dots, Lugol's iodine, methylene blue, crystal violet, fluorescent Dextran, SYTO nucleic acid stains, Alexa Fluor dyes, gold nanoparticles and silver nanoparticles. The one or more markers or contrast agents may include a fluorophore or nanoparticle. The sample may include cells, and the fluorophore or nanoparticle may be targeted for one or more particular cells. Cancer or other diseases or conditions may be diagnosed. Imaging may be done in vivo. Imaging may involve fluorescent and/or reflectance imaging (i.e., separate or together). The method may also include simultaneously imaging the sample with a Magnetic MRI device. The MRI device may be used to navigate imaging with the fibers. Use with an MRI device may include (a) monitoring of delivery and pharmacokinetics of nanoparticle-mediated molecular therapeutics; (b) monitoring of delivery of molecular therapy and an earliest molecular response; or (c) imaging of biomarkers associated with delayed response to molecular therapeutics. The MRI device may be used to monitor a distribution of contrast agents in the sample. The method may also include monitoring interactions of the contrast agents in the sample. Diagnosis of the sample need not include staining of the sample. The method may also include analyzing a margin of the sample using data generated through imaging. Analyzing the margin may include imaging an extent of tumor growth.

In another respect, embodiments of the invention involve a method of imaging in which a patient is identified as being in need of a needle biopsy. The patient is subjected to optical imaging instead of the needle biopsy. The optical imaging may be done as described in this disclosure.

To the extent the term “needle biopsy imaging system” is used to describe embodiments of this invention or aspects of it, one should not interpret the phrase to necessarily suggest a similarity with conventional needle biopsies. This term is employed to indicate that optical techniques of this disclosure can replace or supplement traditional needle biopsy techniques. For example, instead of employing a traditional needle-based system, one may instead use an optical probe, as taught by embodiments of this disclosure, to, e.g., diagnose or monitor a tissue site. In other embodiments, an optical probe may be used in conjunction with a needle from a traditional biopsy imaging system to provide access to tissue.

The term “lensless” when applied to embodiments of this invention refers to the lack of a focusing lens at the distal end (and particularly, a distal tip) of the endoscopic probe. The distal end of the endoscopic probe may comprise other non-focusing lenses, such as a magnifying lens. Other lenses may exist in the apparatus to achieve tasks such as, e.g., focusing laser radiation into a fiber from a source.

The terms “sample emission” and “sample optical signal” when applied to embodiments of this invention refers to a signal (such as a reflection or a fluorescence) emitted from a sample.

The term “image guide” when applied to embodiments of this invention refers to an apparatus capable of transmitting a sample emission from a sample to an optical system that directs the sample emission to a detector.

The terms “a” and “an” are defined as one or more unless this disclosure explicitly requires otherwise.

The term “substantially” and its variations are defined as being largely but not necessarily wholly what is specified as understood by one of ordinary skill in the art, and in one non-limiting embodiment the term substantially refers to ranges within 10%, preferably within 5%, more preferably within 1%, and most preferably within 0.5% of what is specified.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

The term “coupled,” as used herein, is defined as connected, although not necessarily directly, and not necessarily mechanically.

Other features and associated advantages will become apparent with reference to the following detailed description of specific, example embodiments in connection with the accompanying drawings.

### BRIEF DESCRIPTION OF THE DRAWINGS

These drawings are part of the specification. The drawings offer examples but do not limit the invention. Use of the same element numbers indicates an identical, or functionally similar, component. The drawings are not to scale.

FIG. 1 is a schematic diagram of a needle biopsy imaging system in fluorescent mode, in accordance with embodiments of this disclosure.

FIG. 2 is a schematic diagram of a needle biopsy imaging system in reflectance mode, in accordance with embodiments of this disclosure.

FIG. 3 is a schematic diagram of a needle biopsy imaging system in fluorescent mode, in accordance with embodiments of this disclosure.

FIG. 4 is a schematic diagram of a needle biopsy imaging system, in accordance with embodiments of this disclosure.

FIG. 5 is a detailed view of a graded-index lens system.

FIG. 6 is a schematic diagram of a needle biopsy imaging system, in accordance with embodiments of this disclosure.

FIG. 7 is an image of quantum dot labeled cancer cells acquired with a needle biopsy imaging system in accordance with embodiments of this disclosure.

FIG. 8 is an image of fluorescent polystyrene spheres acquired with a needle biopsy imaging system in accordance with embodiments of this disclosure.

FIG. 9 is an image of breast cancer cells acquired with prior art methods and apparatus.

FIG. 10 is an image of breast cancer cells acquired with a needle biopsy imaging system in accordance with embodiments of this disclosure.

FIG. 11 shows images of carcinoma cells acquired with a needle biopsy imaging system in accordance with embodiments of this disclosure.

FIG. 12 shows images of a target guide and cells acquired with a needle biopsy imaging systems in accordance with embodiments of this disclosure.

### DESCRIPTION OF ILLUSTRATIVE EMBODIMENTS

The description below is directed to specific embodiments, which serve as examples only. Description of these particular examples should not be imported into the claims as extra limitations because the claims themselves define the legal scope of the invention. With the benefit of the present disclosure, those having ordinary skill in the art will comprehend that techniques claimed and described here may be modified and applied to a number of additional, different applications, achieving the same or a similar result. The claims cover all such modifications that fall within the scope and spirit of this disclosure.

The techniques of this disclosure can be applied to many different types of applications, including any application involving the imaging of tissue, and more particularly any application that would conventionally call for a needle biopsy.

In a one embodiment, the invention involves a lensless fiber optic endoscope. This endoscope may be used as an optical needle biopsy to image a layer of cells that are in contact with, or substantially in contact with, the distal tip of the endoscope. It may be used to detect the presence of fluorophores that have labeled individual cells. In conjunction with the targeted fluorophores, it represents a powerful tool to immediately, or nearly immediately, detect the presence of, e.g., cancer and other tissue abnormalities in vivo. In other embodiments, analysis may be done in vitro. This technology may be used to image tissue reflectance or other contrast agents as well. In one embodiment, the optic endoscope is MRI compatible (it can be used inside magnets that are commonly used for MRI) and can therefore be used for simultaneous MRI and optical imaging.

In this embodiment, the lack of a focusing lens at the distal end of the endoscopic probe is noteworthy. The probe comes into direct contact with the site to be imaged and relays the image from that site to, e.g., a digital camera for recording. This places the focal plane of the object at, or substantially at, the distal tip of the device and allows confocal-type images to be obtained without the need for background filtering. Eliminating or reducing the need for background filtering, in turn, offers several advantages such as, but not limited to, lower costs.

Another noteworthy characteristic of this embodiment is that it does not require tissue removal for cellular analysis and diagnosis. Typical biopsies, in contrast, are time consuming and work intensive, requiring tissue removal and staining with contrast agents before diagnosis is possible. The fiber optic endoscope device is a simpler and less inexpensive alternative to image tissue reflectance or fluorescence in vivo, in a minimally-invasive procedure. This imaging system is adaptable to image multiple organ sites in the body.

Yet another noteworthy characteristic is this device's ability to eliminate or drastically reduce the multiple waiting periods between suspicion, removal, and diagnosis typical of biopsies. An associated advantage is that treatment, if necessary, can begin much more quickly, and patients will therefore have less anxiety waiting for the results of the diagnostic procedure. With an in vivo imaging system, diagnosis is possible immediately, as opposed to a biopsy that requires significant time to: remove the tissue, histological slice and stain the sample, and analyze the sample at a pathology lab before an ultimate diagnosis can be made.

Embodiments of this disclosure make it possible to monitor, e.g., molecular therapeutics to evaluate treatment efficacy quickly. One problem associated with biopsies is that any given site must heal between biopsies, which makes ongoing treatment monitoring difficult. When disease progression and response to medication needs to be monitored quickly and frequently, minimally invasive procedures are critical, and embodiments described and illustrated here can be used to this end.

As a result of compatibility with MRI imaging techniques, embodiments of this disclosure can be directly correlated with MRI information. For example, MRI be used to guide the device to investigate regions of interest. The device can be used in combination with MRI and MRI/optical bi-functional contrast agents for at least the following: (1) monitoring of delivery and pharmacokinetics of nanoparticle-mediate molecular therapeutics; (2) simultaneous monitoring of delivery of molecular therapy and the earliest molecular response; and (3) imaging of biomarkers associated with delayed response to molecular therapeutics. In these settings, MRI may be used to monitor the distribution of the contrast agents in the entire patient or test subject. Simultaneously, interactions of the contrast agents in the organ site of interest may be visualized in detail using the imaging device.

Yet another application for the imaging system involves margin removal. Frequently during tumor removal, margin detection is difficult and extra tissue is removed to ensure complete excision. This device, however, can be used during surgery to image the extent of tumor growth and guide the surgery. In this way, margin procedures may be greatly improved.

Yet another application involves scientific research. For example, the imaging may be used to assist in the development of contrast agents for in vivo imaging. The small size of the imaging device, in certain embodiments, allows for imaging of small animals that are frequently used in pre-clinical studies for new contrast agents and targeting mechanisms. For example, an endoscope encompassing elements described here may be incorporated into a probe sized to accommodate, e.g., mice. Such a probe can then be used to assess the effectiveness of different contrast agents.

The optical techniques of this disclosure allow for a less invasive procedure than biopsy. Additionally, the device can readily be made to smaller than typical needles used for biopsies of suspicious lesions and tumors. Additionally, the optical nature of the device means that tissue no longer must be removed to perform an analysis.

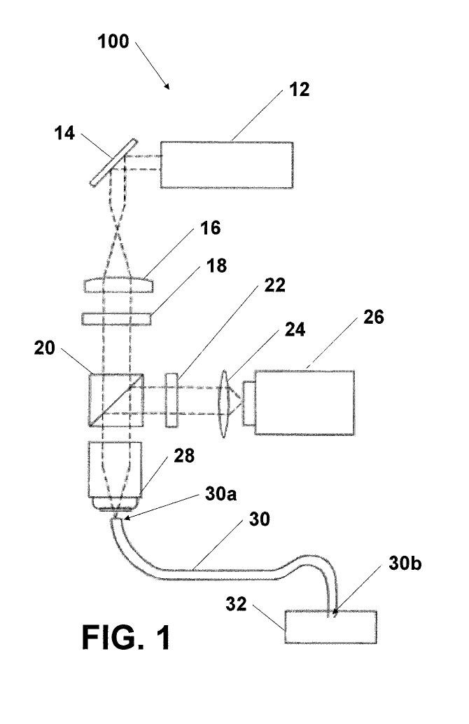

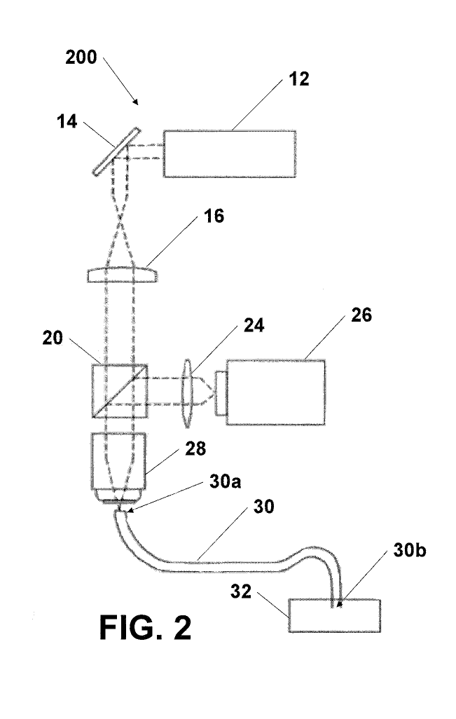

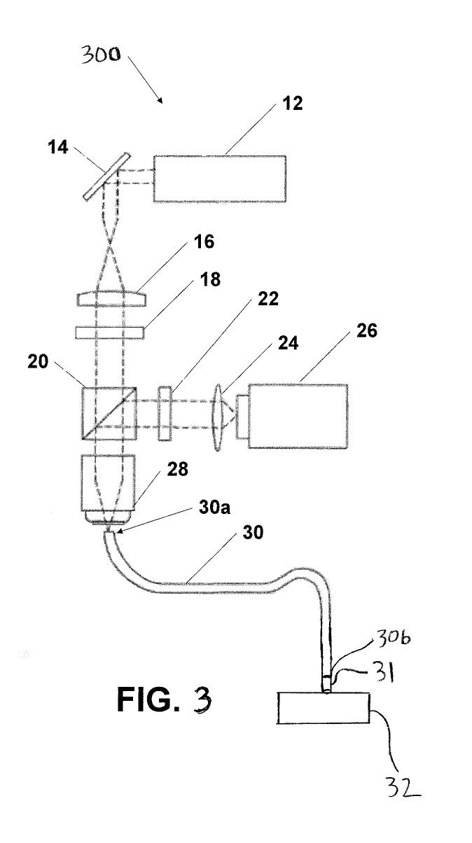

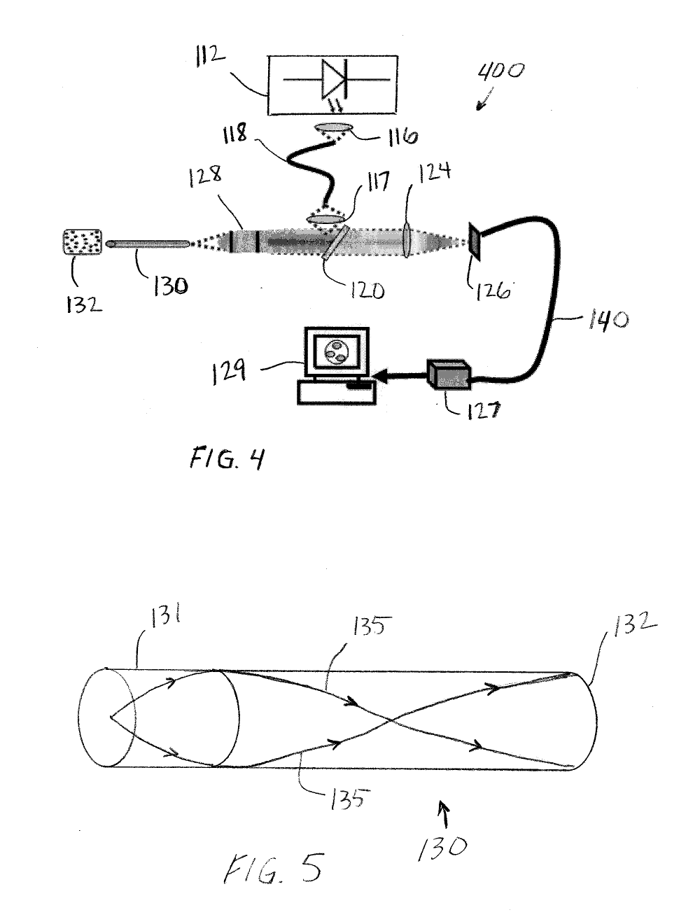

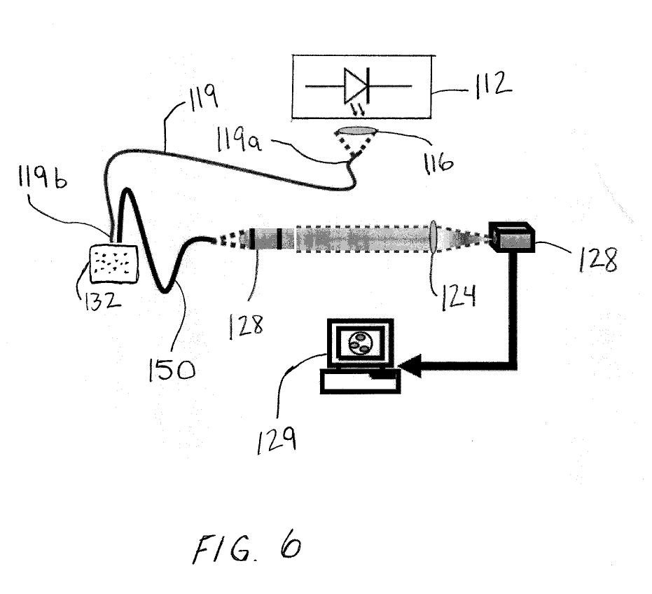

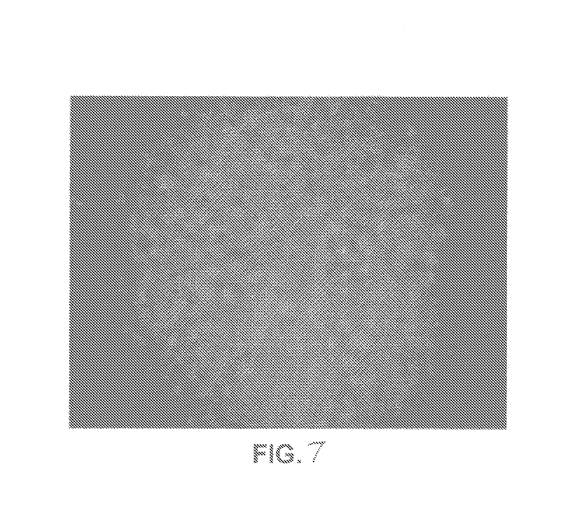

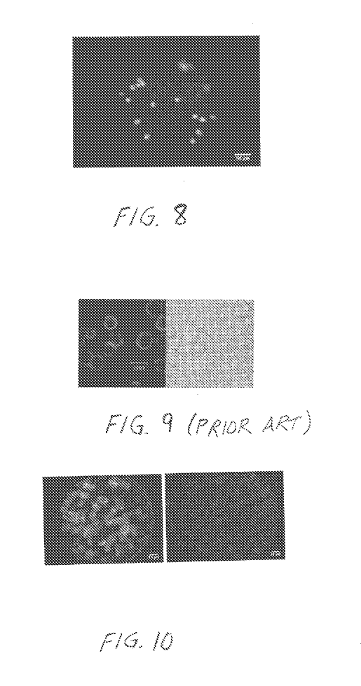

In one embodiment, the endoscope enables imaging at subcellular resolution by way of its physical design. Other in vivo endoscopic imaging systems rely on macroscopic changes in tissue morphology to guide diagnosis. Depending on the fiber(s) that make up an image, the resolution may be limited. In the embodiments described at FIG. 1 and FIG. 2, e.g., the resolution allows for the distinguishing of objects down to about 2 μm. While this may be considered a disadvantage compared to many optical microscopes, it is sufficient for cellular resolution in vivo. Using different fibers or additional components, however, the resolution may be modified. Further, as explained in the discussion of FIG. 3 below, it may be possible to overcome even this resolution limitation with the addition of a GRIN lens to the distal end of the fiber bundle. Resolution may also be enhanced through the use of a GRIN lens system for an image guide in lieu of a fiber bundle, as shown in the discussion of FIGS. 4 and 5.

An application of the cellular resolution of this device involves the detection of cancerous cells labeled with targeted, fluorescence nanoparticles. With other particle functionality, the device's utility may be extended to image many other types of diseases or monitor cellular processes. Embodiments of this disclosure are not tied to a particular fluorescent agent and may be used to do similar imaging with, e.g., many other types of fluorophores or agents known in the art. For example, different embodiments may be used to image metallic, non-fluorescent nanoparticles and, in still other embodiments, even native un-labeled tissue. To the extent any such embodiment may require modification (e.g., modification of the spacing of optical components), such modifications would be well within the grasp of one having ordinary skill in the art.

The reader is directed to the embodiments of FIG. 1 and FIG. 2. These devices include an image guide comprising a coherent fiber optic imaging bundle that is commercially available from a variety of sources. Other portions of these devices involve components to couple light into the fiber bundle, and then to image the light that returns through the fibers. The light source is collimated, passed through a beam splitter and focused onto a proximal end of the fiber bundle. The input light is focused in such a way as to match or closely approximate the numerical aperture of the fibers so that the maximum amount of light will be coupled into the fibers. The light is transmitted through the fibers, out into the tissue of the sample. A portion of the light reflected or generated in the tissue is coupled back into the fibers. Because the numerical aperture (acceptance angle) of the fibers is relatively small, the light generated deep in the tissue, which is scattered multiple times, will have a much smaller chance of re-entering the fibers. The light generated at the fiber surface, which is not scattered, will be more likely to enter the fibers. This fact accounts for the depth sensitivity and narrow depth of field that is exploited in imaging. The returning signal that is accepted into the distal end of the fiber bundle returns through the fibers, is collimated again by the objective lens, is redirected at the beam splitter, and is focused onto a detector such as a CCD camera for imaging.

All of the components illustrated in FIG. 1 and FIG. 2 are fairly common and commercially available. One noteworthy aspect comes in the lack of a focusing lens at the distal tip of the fiber bundle. This limits the field of view to the size of the imaging bundle and limits the resolution to the size of the individual fibers. The lack of a focusing lens at the distal end of the fiber bundle also contributes to the narrow depth of field.

FIG. 1 is a schematic diagram of an example needle biopsy imaging system 100 in fluorescent mode. FIG. 2 is a schematic diagram of an example needle biopsy imaging system 200 in reflectance mode. These embodiments will be described together to the extent that they share several identical or functionally-similar components. The systems include a source 12. This source may be a laser source, a light emitting diode (LED) or other source of radiation sufficient to effect the desired imaging. It may be one or more sources. Mirror 14 is used to direct the source towards the sample. Any optical component for steering a beam or other source of radiation may be used. Lens 16 focuses the source prior to entering one or more fibers. In FIG. 1, excitation filter 18 is used to ensure that radiation sufficient to excite the sample itself or select markers is passed to the one or more fibers. Filters may also be used in FIG. 2 to ensure that the quality of the incident radiation is sufficient to effect a desired imaging characteristic. The source radiation is passed to a beam splitter 20 and further to objective lens 28, which directs the radiation into the one or more fibers illustrated as fiber bundle 30. Objective lens 28 may be any of several commercially available lenses known in the art, and its characteristics may be chosen to optimize coupling and imaging with the one or more chosen fibers. Fiber bundle 30 includes a proximate end 30a and a distal end 30b. The source radiation exits distal end 30b, which lacks a focusing lens. The radiation strikes sample 32 for imaging. For example, in fluorescent mode, the radiation excites the sample itself and/or markers that emit characteristic secondary radiation.

A sample optical signal (either reflectance or fluorescence) from sample 32 enters distal end 30b and travels back through the fiber bundle 30, which serves as an image guide by transferring radiation from sample 32 back through the objective lens 28. In certain embodiments of the present disclosure, a hollow 16 to 18 gauge needle (not shown) can serve as a conduit for the image guide of the endoscope apparatus, thereby providing access to deep tissue.

The sample optical signal from sample 32 then reaches beam splitter 20, which directs the signal towards the detector (shown here as charge-coupled device (CCD) 26). In FIG. 1, the sample radiation passes through emission filter 22, which can be configured to ensure that one or more particular wavelengths reaches the detector. For example, if one is interested in determining whether a particular wavelength (or range of wavelengths) is present in the secondary radiation from the sample, emission filter 22 may be used to block extraneous wavelengths or ranges. Likewise, although not shown, the FIG. 2 device may employ similar filtering techniques to aid in detection of select radiation. Radiation from the sample passes through lens 24, and ultimately onto CCD 26. Other detector types may be used instead of, or in addition to, CCD 26. Signals from CCD 26 are then input to a computer (not shown) or other appropriate data handling device for ultimate analysis and/or storage. Such a data handling device may be integral with, or separate from systems 100 or 200.

The data from CCD 26 may be presented in any number of ways, as is known in the art. For example, a real-time image may be displayed on a computer screen. Images may be stored for later use. Data may be presented graphically or in text form. A report may be generated manually, automatically, or semi-automatically. Such a report may be used to deliver a diagnosis or result.

In the embodiment shown in FIGS. 1 and 2, fiber bundle 30 is produced by Sumitomo Electric Company with the following specifications: an outer diameter of 400 microns, center-to-center spacing of the pixel elements of approximately 4 microns, and a usable field of view of 300 microns. Additionally, the bundle of this embodiment has a 2 cm bend radius, allowing for convenient positioning of the device, and a relatively high numerical aperture of 0.35 to collect as much light as possible. Both ends of fiber bundle 30 may be polished optically flat using 12, 9, 3, and 1 micron lap films using a mechanical fiber optic polisher.

Referring now to FIG. 3, another schematic diagram of an example needle biopsy imaging system 300 is shown in fluorescent mode. The system of FIG. 3 is similar to the system of FIG. 1, with the exception that a graded-index lens (GRIN) lens apparatus 31 has been added to distal end 30b of fiber bundle 30. Although not shown, a GRIN lens apparatus may also be added to the distal end 30b of system 200. GRIN lens apparatus 31 can be coupled to distal end 30b by any manner known to those of skill in the art, such as cementing with optical epoxy. GRIN lens apparatus 31 serves a magnifying lens, rather than a focusing lens. The addition of GRIN lens apparatus 31 improves the resolution of imaging system 300 (as compared to imaging systems 100 or 200) and provides the potential for the distinguishing of objects less than 2 μm.

As explained in more detail below, GRIN lenses have the unique property of a variable refractive index that changes in the radial direction. These cylindrical lenses can be manufactured to very small diameters and are relatively inexpensive to mass produce and simple to align properly. The geometric path of the light rays traveling through these lenses follow a sinusoidal pattern and can be designed to perform in much the same way as standard compound concave or convex glass lens systems.

Referring now to FIG. 4, another schematic diagram of an example needle biopsy imaging system 400 is shown. The embodiment shown in FIG. 4 operates under the same general principles as the previously-described embodiments, but comprises certain differences in components. For example, the image guide of system 400 comprises a GRIN lens apparatus 130, rather than fiber bundle 30 in systems 100 and 200 (or a combination of fiber bundle 30 and GRIN lens apparatus 31 in system 300). An overview of the operation of system 400 is provided below.

System 400 includes a source 112 that emits light or other forms of radiation sufficient to effect the desired imaging. In the embodiment shown, source 112 comprises an LED. LEDs are currently available that have desirable spectral characteristics for certain embodiments (small bandwidth, down to 20 nm FWHM) and illumination intensity (hundreds of milliwatts) and cost only dollars per unit. The ability of such LEDs to function for thousands of hours, as well as inexpensive replacement costs, makes LED's particularly suited for this application. In other embodiments, source 112 may be a laser source and/or may be one or more sources.

Lens 116 is used to direct radiation from source 112 towards a fiber optic light guide 118. In the embodiment shown, fiber optic light guide 118 is a single fiber light guide. In other embodiments, fiber optic light guide may comprise multiple fibers. In still other embodiments, any optical component for steering a light beam or other source of radiation may be used.

Radiation from source 112 (or “source radiation”) exits fiber optic light guide 118 and passes through lens 117 before being directed by dichroic mirror or beam splitter 120 to objective lens 128. Objective lens 128 directs the source radiation to GRIN lens apparatus 130. Objective lens 128 may be any of several commercially available lenses known in the art, and its characteristics may be chosen to optimize coupling and imaging with GRIN lens apparatus 130. Source radiation strikes sample 132 for imaging. For example, in fluorescent mode, the radiation excites sample 132 itself and/or markers that emit characteristic secondary radiation. In reflectance mode, source radiation is reflected off of sample 132. As used herein, a reflection or fluorescence of sample 132 shall be known as a “sample emission” or an “sample optical signal”. As explained in more detail below, it is desirable in certain embodiments to excite quantum dots (semiconductor nanocrystals) and other fluorophores for sample imaging.

A sample emission enters GRIN lens apparatus 130 and travels through objective lens 128 and passes through beam splitter 120. The sample emission then passes through a “tube” lens 124 and is directed towards the detector 126. Collimated light exiting objective lens 128 must be focused onto the plane of detector 126. The focal length of tube lens 124 is therefore important. The focal length of tube lens 124 is directly proportional to the magnification of the optical setup, which is given by the simple relation: Magnification=(focal length of tube lens)/(focal length of objective lens). In one embodiment, the focal length of the objective lens is 18 millimeters, the tube lens focal length is 250 millimeters, and the overall magnification is approximately 14×.

In the embodiment shown, detector 126 is a CCD chip that is electronically coupled to a display device 129 via wiring 140 and control module 127. System 400 may also comprise a computer (not shown) or other appropriate data handling device for ultimate analysis and/or storage. Such a data handling device may be integral with, or separate from systems 400.

The data from detector 126 may be presented in any number of ways, as is known in the art. For example, a real-time image may be displayed on a computer screen. Images may be stored for later use. Data may be presented graphically or in text form. A report may be generated manually, automatically, or semi-automatically. Such a report may be used to deliver a diagnosis or result.

A detailed view of GRIN lens apparatus 130 from system 400 is shown in FIG. 5. As shown, GRIN lens apparatus 130 comprises a magnifying lens 131 and a relay lens 132. Sample emission rays 135 that pass through GRIN lens apparatus 130 follow a generally sinusoidal path as a result of the variable refractive index that changes in the radial direction. In a preferred embodiment, magnifying lens 131 magnifies a portion of sample 132 by a factor of two and relay lens 132 is long enough to allow GRIN lens apparatus 130 to pass through a biopsy needle (not shown) and into a specific tissue of interest.

Referring now to FIG. 6, an alternate embodiment is shown that separates the illumination channel from the imaging path. In the embodiment shown, lens 116 is used to direct radiation from source 112 towards a proximal end 119a of a fiber optic light guide 119. Radiation is emitted from a distal end 119b of fiber optic light guide 119 and strikes sample 132 for imaging. Fiber optic light guide 119 may comprise one or more optical fibers. In preferred embodiments, distal end 119b is proximal to sample 132 and image guide 150. In the embodiment shown, image guide 150 comprises optical fibers; in other embodiments, image guide 150 may comprise a GRIN lens apparatus similar to the image guide of FIG. 3 or FIG. 4. A sample emission from sample 132 passes through image guide 150 and enters objective lens 128. The sample emission then passes through tube lens 124 and is directed towards detector 128 and transferred to display device 129.

Embodiments of the present disclosure can be used for both fluorescence and reflectance image with minor changes in the optical setup. For example, in a preferred embodiment, fluorescence imaging utilizes a dichroic mirror in the objective lens pathway and both an excitation and emission edge-pass filter placed in front of the source and the detector respectively. This setup effectively illuminates the proximal face of the image guide and eliminates remaining excitation light before entering the detector. For embodiments utilizing reflectance imaging, the edge-pass filters may be removed and the dichroic mirror replaced with a polarizing beam splitter cube (PBS). This PBS cube splits the incoming unpolarized light to s- and p-polarizations, and sends only one to the proximal face of the image guide. Specular reflection from this surface will remain polarized in the direction that is unfavorable to be passed through to the detector. In certain embodiments, the distal end of the fiber bundle can be polished at a 10-degree angle, which reduces the amount of reflected light from this surface by directing it outside the acceptance cone of the individual fibers in the image guide. In certain embodiments, the acceptance cone is defined by the fiber numerical aperture of approximately 0.35.

As previously mentioned, certain embodiments of the present disclosure employ quantum dots for sample imaging. Quantum dots are fluorescent crystals that have several highly advantageous properties for biological imaging: a broad excitation profile, a narrow, tunable emission profile, limited photobleaching, and the ability to be passivated and functionalized to accept targeting antibodies on their surfaces. Quantum dots also exhibit a high quantum efficiency and a large Stokes shift, meaning that relatively little excitation light would be required to generate a signal, and this excitation light is easily filtered from the emitted light. Quantum dots and quantum dot-antibody conjugates are also a satisfactory size for labeling tissues—usually between 2 to 10 nm. These small sizes allow for the particles to pass through tissues and contact cell membranes, allowing the particles to be used to label intracellular targets. Quantum dots have been used extensively recently to label cultures of tumor cells in vitro as well in small animal models for cancer imaging related studies.

Targeting of quantum dot fluorescent markers with the aid of antibodies or aptamers should allow clinicians to monitor the expression levels of extracellular receptors like Her-2 and EGFR (epidermal growth factor receptor). Drugs such as Trastuzumab and Cetuximab block Her-2 and EGFR receptors and have been shown to be beneficial in breast cancers that express those receptors. By directly visualizing these receptors over the course of treatment, it may be possible to track the progression and response of cancers to treatment.

Quantum dots have desirable absorption cross sections such that illumination over a broad wavelength range in the blue region of the spectrum would be sufficient, eliminating the need for a laser or arc lamp, the standard methods for excitation of many fluorescent molecules. In certain embodiments, the source is a Luxeon III Star LED from Lumileds Corp., which emits 400 milliwatts of power at a peak emission wavelength of 455 nm with a 20 nm FWHM. For other applications, such as reflectance imaging, this wavelength can be easily and quickly changed by simply changing out the LED module.

As described herein, embodiments of the present invention can be utilized to image samples without some of the issues associated with previous systems and techniques.

### EXAMPLES

The following examples are included to demonstrate aspects of specific experiments related to this disclosure. FIGS. 7-12 present data associated with embodiments of this disclosure. Subject matter presented as an example may be encompassed by the present claims or added to the claims to define protected subject matter.

FIG. 7 shows quantum dot (Qdot 655 nm) labeled cancer cells with broadband excitation and long pass filter at 620 nm, imaged with a needle biopsy system as described herein.

FIG. 8 shows 15 micron diameter fluorescent polystyrene spheres (produced by Invitrogen) in a 10% gelatin phantom. This image was acquired with a needle biopsy system as described here comprising a 455 nm peak emission LED (produced by Lumileds) and a 500 nm long pass filter (produced by Thorlabs).

FIG. 9 shows images of SK-BR-3 (a breast cancer cell line, from American Type Culture Collection)breast cancer cells labeled with anti-Her-2 antibody (Neomarkers) and 585 nm emission quantum dots (Invitrogen). Her-2 is a tyrosine kinase-class receptor in the cell membrane of many cells, and highly overexpressed in some types of breast cancer. It is the target of the recently approved drug Herceptin. The image on the left side was captured using fluorescent confocal imaging techniques incorporating a Zeiss LSM 510 confocal microscope was using an excitation wavelength of 458 nm and a Long Pass filter cutoff of 505 nm. The image on the right side was produced utilizing Differential Interference Contrast (DIC).

FIG. 10 shows an image of anti-Her-2 and 585 nm quantum dot labeled SK-BR-3 cells suspended in a collagen phantom on the left and an image of the same preparation using an isotype control antibody on the right. Each of the images was acquired with needle biopsy system incorporating a fiber bundle image guide.

FIG. 11 shows images of Toluidine blue labeled squamous carcinoma cells on a monolayer of collagen. Images were collected using an embodiment configured with a separate illumination channel, and demonstrate the contrast induced by collection of backscattered light.

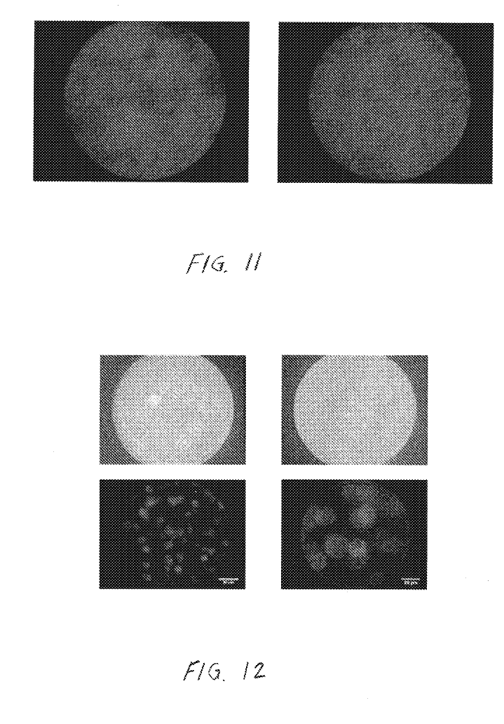

FIG. 12 demonstrates the improved spatial resolution that can be achieved with the addition of a GRIN lens system to the image guide. The images on the left were obtained with an embodiment incorporating a fiber optic image guide without a GRIN lens system. The images on the right were obtained with an embodiment utilizing a fiber optic image guide incorporating a 2× GRIN lens (manufactured by Grintech) coupled to the distal end of the image guide. The top row of images demonstrates the resulting improved resolution and decreased field of view on a USAF resolution target. The bottom row demonstrates the effect on anti-EGFR and 655 nm quantum dot labeled 1483 cells in a collagen phantom. Below is a schematic of the proposed GRIN-lens based microscope system.

With the benefit of the present disclosure, those having ordinary skill in the art will recognize that techniques claimed here and described above may be modified and applied to a number of additional, different applications, achieving the same or a similar result. The attached claims cover all such modifications that fall within the scope and spirit of this disclosure.

### REFERENCES

Each of the following references is incorporated by reference in its entirety:
1. U.S. Pat. No. 6,766,184
2. U.S. Pat. No. 6,697,666
3. U.S. Pat. No. 6,639,674
4. U.S. Pat. No. 6,593,101
5. U.S. Pat. No. 6,571,118
6. U.S. Pat. No. 6,370,422
7. U.S. Pat. No. 6,258,576
8. U.S. Pat. No. 6,241,662
9. U.S. Pat. No. 6,187,289
10. U.S. Pat. No. 6,135,965
11. U.S. Pat. No. 6,095,982
12. U.S. Pat. No. 5,991,653
13. U.S. Pat. No. 5,929,985
14. U.S. Pat. No. 5,920,399
15. U.S. Pat. No. 5,842,995
16. U.S. Pat. No. 5,699,795
17. U.S. Pat. No. 5,697,373
18. U.S. Pat. No. 5,623,932
19. U.S. Pat. No. 5,612,540
20. U.S. Pat. No. 5,562,100
21. U.S. Pat. No. 5,421,339
22. U.S. Pat. No. 5,421,337
23. U.S. Pat. No. 5,419,323
24. U.S. Pat. No. 5,345,941
25. U.S. Pat. No. 5,201,318

1. An imaging system for imaging a tissue sample, comprising:
an image guide having a plurality of coherent optical fibers, the image guide having a proximate end and a lensless distal end, wherein a focal plane of the image guide is substantially at a transverse tip of the distal end;
a radiation source;
optics configured to direct radiation from the source to the proximate end of the fiber bundle;
a detector; and
optics configured to direct a sample emission from the proximate end of the fiber bundle to the detector.

2. The imaging system of claim 1, wherein the imaging system is configured to image a sample in vivo.

3. The imaging system of claim 1, wherein the imaging system is configured to operate in a fluorescent mode.

4. The imaging system of claim 1, wherein the imaging system is configured to operate in a reflectance mode.

5. The imaging system of claim 1, wherein the imaging system is compatible with a Magnetic Resonance Imaging (MRI) device.

6. The imaging system of claim 1, wherein the resolution of the imaging system is about 2 micrometers.

7. The imaging system of claim 1, wherein the image guide contains a first set of fibers configured to transmit the radiation from the source to the tissue sample and a second set of fibers configured to transmit the sample emission from the tissue sample to the detector.

8. The imaging system of claim 1, wherein the image guide contains a plurality of fibers configured to both transmit the radiation from the source to the tissue sample and to transmit the sample emission from the tissue sample to the detector.

9. An imaging endoscope apparatus comprising:
an image guide having a plurality of coherent optical fibers, the image guide having a proximate end and a lensless distal end, wherein a focal plane of the image guide is substantially at a transverse tip of the distal end;
a radiation source;
a focusing lens configured to direct radiation from the source to the proximate end of the fiber bundle;
an image capture device that detects an image of a plane of a radiated tissue site that is in contact with the tip of the distal end;
an emission lens configured to direct a sample emission from the proximate end of the image guide to the image capture device; and
a beam splitter in optical communication with the proximate end of the image guide, the focusing lens, and the emission lens.

10. The apparatus of claim 9, wherein the detector is a CCD detector.

11. The apparatus of claim 9, wherein the apparatus further comprises an excitation filter or an emission filter, the apparatus being configured to operate in a fluorescent mode.

12. The apparatus of claim 9, wherein the apparatus is configured to operate in a reflectance mode.

13. The imaging system of claim 9, wherein the image guide contains a first set of fibers configured to transmit the radiation from the source to the tissue sample and a second set of fibers configured to transmit the sample emission from the tissue sample to the detector.

14. The imaging system of claim 9, wherein the image guide contains a plurality of fibers configured to both transmit the radiation from the source to the tissue sample and to transmit the sample emission from the tissue sample to the detector.

15. An imaging endoscope apparatus comprising:
an image guide having a plurality of coherent optical fibers with substantially identical numerical apertures, wherein the image guide has a proximate end and a distal end, and wherein a focal plane of the image guide is substantially at a transverse tip of the distal end;
a source of fluorescent radiation;
a detector;
a beam splitter;
a collimating lens and an excitation filter configured to direct radiation from the source through the beam splitter to the proximate end of the image guide; and
an emission filter configured to filter a sample emission being transmitted from the proximate end of the image guide through the beam splitter to the detector.

16. The apparatus of claim 15, wherein the collimating lens focuses the source radiation to substantially match the numerical aperture of the optical fibers.

17. The apparatus of claim 15, wherein the detector is an image capture device that detects an image of a plane of a radiated tissue site in contact with the distal end of the image guide.

18. An imaging endoscope apparatus comprising:
an image guide having a plurality of coherent optical fibers, the image guide having a proximate end and a transverse distal end;
a graded-index lens system having a first and a second transverse end, wherein the first transverse end is optically mated to the transverse tip of the distal end of the image guide and wherein a focal plane is at the second transverse end;
a source of radiation;
a detector;
a beam splitter;
a focusing lens configured to direct radiation from the source through the beam splitter to the proximate end of the image guide; and
an emission lens configured to direct a sample emission from the proximate end of the image guide through the beam splitter to the detector.

19. The apparatus of claim 18, wherein the graded-index lens system comprises a magnifying lens and a relay lens.

20. The apparatus of claim 18, wherein the optical fibers have substantially identical numerical apertures and the collimating lens focuses the source radiation to substantially match the numerical aperture of the optical fibers.

21. The apparatus of claim 18, wherein the detector is an image capture device that detects an image of a plane of a radiated tissue site in contact with the focal plane of the second transverse end of the graded-index lens system.

22. The apparatus of claim 18, wherein the apparatus further comprises an excitation filter or an emission filter, the apparatus being configured to operate in a fluorescent mode.

23. The apparatus of claim 18, wherein the apparatus is configured to operate in a reflectance mode.
